(19) United States
(12) Patent Application Publication
(10) Pub. No.: US 2010/0249747 A1
(43) Pub. Date: Sep. 30, 2010

(54) Transdermal venous access locking solution

(75) Inventors: Stanley L. Mills, Goldsby, OK (US); Jacqueline L. Mills, Goldsby, OK (US); Robert D. Maurer, Elkhorn, NE (US); Gary L. Rayburn, Norman, OK (US); Marvin A. Cuchens, Madison, MS (US)

Correspondence Address: Richard G. Gervase;Mintz Levin Cohn Ferris Glovsky and Popeo PC, Chrysler Center, 666 Third Avenue, 24th Floor, New York, NY, 10017, US

(73) Assignee: Organic Medical Ventures, L.L.C., Norman, OK (US)

(21) Appl. No.: 12/383,722

(22) Filed: Mar. 26, 2009

(51) Int. Cl. A61M 25/00; A61K 31/727; A61K 38/17; A61P 7/02; A61K 31/20; A01N 37/00; A61P 31/00

(52) U.S. Cl. 604/506; 514/56; 514/12; 514/558; 514/557; 604/265

(57) ABSTRACT

Compositions and methods of employing compositions in flushing and coating medical devices are disclosed. The compositions include combinations of a chelating agent, anticoagulant, or antithrombotic agent, with a C₄-C₉ carboxylate antimicrobial agent, such as octanoic acid. Methods of using these compositions for coating a medical device and for inhibiting catheter infection are also disclosed. Particular combinations of the claimed combinations include, for example, octanoic acid or other C₄-C₉ carboxylate antimicrobial agent together with EDTA, EGTA, DTPA, heparin and/or hirudin in a pharmaceutically acceptable diluent.

### FIELD OF THE INVENTION

This invention relates to the field of transdermal indwelling medical devices, such as catheters, as well as to the field of methods and compositions for flushing, locking and coating these medical devices. The field of this invention also relates to microbial-inhibiting pharmaceutical preparations. This invention also relates to pharmaceutical preparations useful in maintaining catheter patency and preventing infection. Methods of using the pharmaceutical preparation of the invention in the management and maintenance of transdermal vascular access catheters are also related to the present disclosure.

### BACKGROUND OF THE INVENTION

Transdermal medical devices, including vascular catheters, have become essential in the management of hospitalized or chronically ill patients. Unfortunately, vascular catheters have become the major source for hospital-acquired sepsis. Hence, the benefit derived from transdermal medical devices such as vascular catheters is often upset by infectious complications. Thrombotic occlusions of the lumen of central venous catheters (CVC) are another complication that will often lead to the removal of catheters.

To reduce problems associated with thrombus formation, it is now common to “lock” intravascular access catheters between successive uses. Locking typically involves first flushing the catheter with saline to remove blood, medications, cellular debris and other substances from the catheter lumen. After the catheter has been flushed, a locking solution, typically heparin, is then injected to displace the saline and fill the lumen. The heparin locking solution both excludes blood from the lumen and actively inhibits clotting and thrombus formation within the lumen. To address infection, various antimicrobial substances have been combined with the locking solution in order to inhibit infection at the same time that thrombosis is being inhibited. However, problems with current and continuously emerging resistance to antimicrobial substances, as well as the over-use (and hence the increased risk of developing resistance) of antimicrobials, is an ever-growing concern.

Staphylococcus epidermidis and S. aureus account for 75% of CVC related infections. Candida species account for another 10% to 15% of such infections. The use of antistaphylococcal antibiotics to prevent these infections has been found to reduce CVC related bacterial infections, but only at the expense of the occurrence of higher rates of fungal (Candida) infections. The fibrous glycocalyx material produced by staphylococci and Candida helps these organisms adhere and stick to catheter surfaces. These microbiological biofilm layers are made of fibrous glycocalyx material primarily polysaccharide in nature. The protective sheath provided by the glycocalyx at the infected site effectively prevents the elimination and treatment of these infections. As a result, pharmaceutical preparations are needed that are effective for reducing or eliminating glycocalyx of infectious microorganisms typically associated with catheter colonization and infection.

Transdermal vascular catheters get engulfed by a fibrin sheath that subsequently acts to cover the internal and external surfaces of a catheter. This fibrin sheath provides such organisms as Staphylococci and Candida, with an enhanced adherence capacity to the catheter surface. Unlike these particular microbes, gram-negative bacilli do not adhere well to fibrin and fibronectin. A composition that halts fibrin formation would thus be particularly useful in halting the colonization of Staphylococci, Candida, and the like, at transdermal catheter sites.

Ethylenediaminetetraacetic acid (EDTA) is an anticoagulant used in blood collection tubes. It is also recognized as a calcium chelating agent. EDTA is also recognized to have an antibacterial and antistaphylococcal effect (alone or in combination) (Harper & Epis (1987) Microbios. 51:107; Said et al. (1987) J. Med. Microbiol. 24:267; Root et al. (1988) Antimicrob. Agents Chemother. 32:1627). While those investigators found EDTA to be bacteriocidal, no remedy or suggestion of how the microbial glycocalyx of a device-related infection could be eliminated was provided.

EGTA (ethylene glycol-bis-[beta-aminoethyl ether]-N,N,N′,N′-tetraacetic acid) is another recognized chelating agent. This agent has not been described as antimicrobial. Triethylene tetramine dihydrochloride (trientine 2HCl) (TTH) is a recognized chelating agent that chelates copper. TTH and other chelating agents, including diethylenetriamine pentaacetic acid (DTPA), are similarly not recognized as having antimicrobial activity.

Although glycopeptide antibiotics (vancomycin and teicoplanin) are effective against staphylococci in vitro and in tissue, they are not active against adherent staphylococci embedded in a biofilm layer, such as glycocalyx. While flushing with such agents may acutely destroy these microorganisms, the risk of rapid development of tolerant and resistant strains in the patient being treated makes this a contraindicated procedure in most cases.

U.S. Pat. No. 5,688,516 to I. Radd, describes compositions for use with catheters that include a tetracycline antibiotic, such as minocycline and EDTA.

U.S. Pats. No. 4,343,788 and 4,479,795 to R. V. Mustacich, describe polymer compositions containing carboxylate antimicrobial agents for incorporation into catheters. U.S. Pat. No. 4,392,848 to D. S. Lucas, describes polymer compositions for incorporation into catheters that are permeable to carboxylate antimicrobial agents. U.S. Pat. No. 4,489,097 to R. L. Stone (hereinafter “Stone”), describes intravenous solutions containing carboxylate antimicrobial agents, preferably n-hexanoic and n-octanoic acids and pharmaceutically-acceptable, water-soluble salts thereof.

A prophylactic agent for catheter maintenance should both inhibit/eliminate the formation of polysaccharide-rich glycocalyx and eliminate staphylococci and fungi.

In view of the foregoing, there is a need for improved compositions, kits and methods for flushing, locking and disinfecting catheters. Such compositions should have antimicrobial activity against a broad spectrum of microorganisms, preferably including fungi and both gram-positive and gram-negative bactertia, and preferably be effective against planktonic (free-floating) and adherent microorganisms embedded in a biofilm. The compositions should discourage the development of resistant microbes, be relatively inexpensive, non-toxic, compatible with the catheter material, safe if inadvertently infused systemically, easy to implement, require minimum or no preparation, and be useful with most or all types of implanted catheters, including hemodialysis and hemofiltration catheters, IV catheters, peritoneal dialysis catheters, urinary catheters, chemotherapy catheters, and the like. At least some of these objectives are met by embodiments of the invention described hereinafter.

### SUMMARY OF THE INVENTION

Embodiments of the present invention provides unique and effective pharmaceutical compositions that include effective amounts of a C₄-C₉ carboxylate antimicrobial agent, such as a C₄-C₉ carboxylate antimicrobial agent or antifungal agent, and a chelating agent, anticoagulant or antithrombotic agent. In one preferred embodiment, the chelating agent is EDTA and the C₄-C₉ carboxylate antimicrobial agent is octanoic acid. In other embodiments, the composition comprises a C₄-C₉ carboxylate antimicrobial agent and an anticoagulant, an antithrombotic agent, or a chelating agent other than EDTA. A preferred combination includes a C₄-C₉ carboxylate antimicrobial agent and a calcium chelating agent, such as EGTA. Chelating agents that may be used in conjunction with the present invention include, but are not limited to, EDTA (ethylenediaminetetraacetic acid), EGTA, DTPA (diethylenetriamine pentaacetic acid), DMSA, deferoxamine, Dimercaprol, edetate calcium disodium, triethylene tetramine dihydrochloride, ascorbic acid, zinc citrate, combination of bismuth and citrate, penicillamine, succimer and Editronate. Preferred chelating agents include those that chelate divalent metal cations such as Ca, Mg, Mn, Fe and Zn. In some preferred embodiments, the chelating agent complexes calcium weakly, if at all, e.g., ascorbic acid.

The foregoing compositions can also include a pharmacologically acceptable carrier solution, such as water, Ringers solution or saline pH adjusted to 5.2 or less. The compositions herein have an in-use pH of about 6.0, or below, generally in the range of about 3.5 to about 5.8, most preferably in the pH range of about 3.5 to about 5.2. Within this acidic pH range, proper concentrations of the carboxylate compounds in the free acid form quickly and efficiently kill a wide variety of bacteria and fungi.

The chelating agent of the compositions preferably provides potent glycocalyx inhibiting potential. C₄-C₉ carboxylate antimicrobial agents of the compositions, such as octanoic acid at high concentrations, preferably have a fungicidal effect and a unique ability to penetrate a polysaccharide-rich glycocalyx biofilm layer. The combination of the C₄-C₉ carboxylate antimicrobial agent and chelating agent provides a unique combination anticoagulant, anti-microbial, glycocalyx inhibiting, antibacterial and antifungal agent for the prevention of thrombogenesis, microbial adherence and device-related infections. Octanoic acid-EDTA (O-EDTA) is one example of such a combination that may be preferred for use in a kit. Chelating agents other than EDTA that are desired include in particular EGTA and ascorbic acid.

In other preferred embodiments, the compositions comprise an anticoagulant and an antimicrobial agent, preferably a C₄-C₉ carboxylate antimicrobial agent and most preferably n-octanoic acid. Preferred anticoagulants include heparin, low molecular weight heparin, a combination of citrate and heparin, enoxaparin sodium, coumarin and indanedione derivative, anisindione, warfarin, protamine sulfate, streptokinase, urokinase, anti-thrombin and atlephase recombinant, anistreplase. Another preferred anticoagulant is hirudin. By way of example, useful C₄-C₉ carboxylate antimicrobial agent include n-octanoic, n-butyric. n-pentanoic, n-hexanoic, n-heptanoic, n-nonanoic acids and water-soluble, pharmaceutically-acceptable salts thereof.

Other embodiments of the compositions can include a combination of a chelating agent, a C₄-C₉ carboxylate antimicrobial agent, and heparin. An example of such a combination is EDTA, n-octanoic acid, and heparin.

In another aspect, embodiments of the invention provide methods of using compositions of the chelating agent, anticoagulant or antithrombotic agent mixture with C₄-C₉ carboxylate antimicrobial agents in a variety of therapeutic applications. One such therapeutic application is for preventing catheter infections. An example of a composition to be used in the practice of these methods comprises octanoic acid together with a chelating agent, anticoagulant or antithrombotic agent. EDTA is an example of a chelating agent contemplated for use in these methods; however, other chelating agents would also be expected to be useful.

Particularly preferred preparations of the present invention comprise a mixture of a pharmacologically effective amount of octanoic acid and EDTA, EGTA, triethylene tetramine dihydrochloride, DTPA, hirudin, or heparin in a pharmacologically acceptable carrier solution, either alone or together with other antimicrobials.

For use in maintaining catheter patency, the pharmaceutical preparation of the invention may be efficaciously used with medical devices such as a central venous catheter, a peripheral intravenous catheter, an arterial catheter, a Swan-Ganz catheter, a hemodialysis catheter, an umbilical catheter, a percutaneous nontunneled silicone catheter, a cuffed tunneled central venous catheter, as well as with a subcutaneous central venous port.

Embodiments of the invention also provide medical devices, such as catheters, that are coated with any of the foregoing mixtures. The mixture in one preferred embodiment comprises EDTA and octanoic acid. Where the chelating agent is other than EDTA, the mixture in one example includes EGTA together with an antimicrobial agent, such as a n-octanoic acid. Particular exemplary medical devices that may be prepared and coated with the preparations of the present invention are provided in the above list.

Embodiments of the present invention also provide processes for preparing coated medical devices with the compositions described herein. In a one embodiment, the process comprises exposing the medical device to a composition of a chelating agent, an anticoagulant, or antithrombotic agent combined with a C₄-C₉ carboxylate antimicrobial agent for a sufficient amount of time to provide a coating on the exposed surface of the device. Where the composition is in a liquid form, it can be allowed to dry on the device surface to form a film.

In a preferred embodiment of the above described processes, the device is first treated with a surfactant before exposing the device to the composition. Such surfactants, by way of example, include tridodecylmethyl ammonium chloride and benzalkonium chloride.

For the herein described uses, a combination of a chelating agent and a C₄-C₉ carboxylate antimicrobial agent, particularly n-octanoic, n-butyric, n-pentanoic, n-hexanoic, n-heptanoic, n-nonanoic acids and water-soluble, pharmaceutically-acceptable salts thereof. The concentration of the chelating agent in embodiments of the combination is between about 0.001 to about 1,000 mg/mL, or preferably between about 1 to about 200 mg/mL, or from about 10 to about 100 mg/mL, or preferably between about 20 to about 100 or about 20 to about 60 mg/mL. The concentration of the antimicrobial agent is preferred to be between about 1.0 micromolar to about 1.0 molar or between about 1.0 millimolar to about 200 millimolar, or even more preferably between about 2.0 millimolar to about 100 millimolar or between about 2 millimolar to about 5 millimolar, in the preparation. Most preferably, the combination includes about 30 mg/mL of the chelating agent and about 3.5 millimolar of the C₄-C₉ carboxylate antimicrobial agent.

Where n-octanoic acid is the antimicrobial agent of choice, it can be reconstituted to an appropriate concentration from a vial of n-octanoic acid and then combined in the manner described herein to provide a preparation with the concentration of octanoic acid desired according to methods well known to those of ordinary skill in the art of pharmaceutical preparations. The carrier solution, by way of example, can comprise saline, phosphate buffered saline, dextrose in water, Ringers solution or water pH adjusted to 5.2 or less.

In another aspect, a catheter flushing pharmaceutical preparation is provided. Most preferably, the catheter flushing pharmaceutical preparation comprises a glycocalyx inhibiting concentration of a chelating agent, anticoagulant or antithrombotic agent, and an effective amount of a C₄-C₉ carboxylate antimicrobial agent, in a pharmaceutically acceptable carrier solution (e.g., saline pH adjusted to 5.2 or less). More specifically, the concentration of the chelating agent in embodiments of the preparation is between about 0.001 mg/mL to about 1,000 mg/mL, or between about 1 to about 200, or even more preferably between about 10 to about 100 mg/mL. The concentration of the antimicrobial agent is preferred to be between about 1.0 micromolar to about 1.0 molar or between about 1.0 millimolar to about 200 millimolar, or even more preferably between about 2.0 millimolar to about 100 millimolar or between about 2 millimolar to about 5 millimolar, in the preparation. In one preferred embodiment of the preparation, the chelating agent is EGTA and the C₄-C₉ carboxylate antimicrobial agent is the antimicrobial, n-octanoic acid.

Another embodiment of the catheter flushing pharmaceutical preparation of the invention includes about 30 mg/mL EDTA and about 3.5 millimolar n-octanoic acid. By way of example, the carrier solution is saline, water, or a Ringers solution pH adjusted to 5.2 or less. The catheter flushing preparation of the present invention may advantageously be used to inhibit the formation of polysaccharide-rich glycocalyx. In this manner, infections characterized by such a formation may be effectively eliminated.

Another aspect of the present invention provides a method of preparing a biofilm-resistant medical device. In one embodiment, the method comprises exposing a device with the compositions or catheter flushing preparations described herein. Any of a variety of catheters may be treated or coated according to the described method employing coating techniques well known to those of ordinary skill in the art.

While the method may be used to coat virtually any surface where glycocalyx formation is to be desirably inhibited, use of the method in preparing a microbial biofilm-resistant catheter device is particularly envisioned. By way of example, catheters that may be prepared and treated according to embodiments of the invention include a central venous catheter and a triple lumen catheter. It is anticipated that the method will provide a device resistant to polysaccharide-rich glycocalyx formation, such as that typical of Staphylococci.

In a preferred aspect of the described method, a biofilm-resistant medical device is prepared using a pharmaceutical preparation of a chelating agent, anticoagulant, or antithrombotic agent and a C₄-C₉ carboxylate antimicrobial agent. An example of such preparation comprises a combination of n-octanoic acid and EDTA, or a combination of a chelating agent other than EDTA, antithrombotic or anticoagulant agent together with a C₄-C₉ carboxylate antimicrobial agent. The various concentration ranges of the C₄-C₉ carboxylate antimicrobial agents and chelating agents described above are also contemplated as useful in the compositions for coating a medical device. Other concentration ranges include between about 1.0 micromolar to about 1.0 molar or between about 1.0 millimolar to about 200 millimolar, or even more preferably between about 2.0 millimolar to about 100 millimolar or between about 2 millimolar to about 5 millimolar, in the preparation of the C₄-C₉ carboxylate antimicrobial agent and between 10 mg/mL and about 200 mg/mL of the chelating agent, anticoagulant, or antithrombolic agent. One embodiment of the method comprises use of a composition that includes about 60 millimolar of the C₄-C₉ carboxylate antimicrobial agent and about 60 mg/mL of the chelating agent. Antimicrobial agents that are specific examples for use in these methods include n-octanoic, n-butyric, n-pentanoic, n-hexanoic, n-heptanoic, n-nonanoic acids and water-soluble, pharmaceutically-acceptable salts thereof.

In one aspect, the method comprises preparing a pharmaceutical preparation of the desired combination in a biocompatible adherent coating carrier solution. The surface of the medical device of interest is then exposed to the pharmaceutical preparation for a period of time sufficient to allow the formation of a film or coating of the preparation on the surface of the device. This may be accomplished, for example, by dipping the device in the preparation. Most preferably, the device to be coated is a catheter. Such treatment provides a biofilm-resistant catheter.

The pharmaceutical preparation of the method in a particularly preferred embodiment comprises about 3.5 millimolar of the antimicrobial agent, such as n-octanoic acid, and about 30 mg/mL of the chelating agent or anticoagulant, such as EDTA, EGTA or DTPA.

Embodiments of the present invention also provide methods for inhibiting glycoprotein-rich glycocalyx formation at a catheter port. The method in one embodiment comprises flushing the catheter periodically with a pharmaceutical preparation comprising a glycocalyx-inhibiting concentration of a chelating agent, an anticoagulant or an antithrombotic agent, and a C₄-C₉ carboxylate antimicrobial agent, in a pharmacologically acceptable carrier solution. In one aspect of the method, the composition includes a chelating agent selected from EDTA, EGTA, DTPA or TTH. In particular embodiments, the chelating agent is included in the composition at a concentration of between about 0.001 to about 1,000 mg/mL, or preferably between about 1 to about 200, or between about 10 to about 100 mg/mL. In preferred embodiments, between about 20 to about 60 mg/mL of the chelating agent is included in the flushing solution. A preferred concentration of the chelating agent in the composition is about 30 mg/mL. Where n-octanoic acid is the C₄-C₉ carboxylate antimicrobial agent, a glycocalyx inhibiting concentration can be between about 1.0 micromolar to about 1.0 molar or between about 1.0 millimolar to about 200 millimolar, or even more preferably between about 2.0 millimolar to about 100 millimolar or between about 2 millimolar to about 5 millimolar, in the preparation. A preferred concentration of antimicrobial agent to use is about 3.5 millimolar. Other C₄-C₉ carboxylate antimicrobial agents may also be used, such as n-butyric, n-pentanoic, n-hexanoic, n-heptanoic, n-nonanoic acids and water-soluble, pharmaceutically-acceptable salts thereof.

The described methods can be used to inhibit infection at virtually any tunneled or untunneled catheter. As part of a catheter maintenance regimen, the catheter most preferably is to be flushed with a composition comprising a C₄-C₉ carboxylate antimicrobial agent and a chelating agent, anticoagulant or antithrombotic agent, in a pharmaceutically acceptable carrier solution. The described regimen is repeated once a week, once every 4 days, once every 2 days, once a day (about every 24 hours), twice a day, every four hours or as needed according to patient needs.

In still another aspect, embodiments of the invention provide methods for eliminating microbial glycocalyx formation, particularly polysaccharide-rich (Staphylococcal) glycocalyx formation, at a catheter lumen. The method, in one embodiment, comprises preparing a solution comprising a chelating (such as EDTA, EGTA, or both), anticoagulant or antithrombotic agent, together with a C₄-C₉ carboxylate antimicrobial agent, (such as n-octanoic, n-butyric, n-pentanoic, n-hexanoic, n-heptanoic and n-nonanoic acids), in a carrier solution to provide a flushing composition, and flushing the catheter with a therapeutically effective amount of the flushing composition.

In one embodiment, the flushing composition is an O-EDTA preparation that includes a concentration of octanoic acid of between about 1.0 micromolar to about 1.0 molar or between about 1.0 millimolar to about 200 millimolar, or even more preferably between about 2.0 millimolar to about 100 millimolar or between about 2 millimolar to about 5 millimolar, in the preparation and a concentration of EDTA of between about 10 to about 100 mg/mL (preferably between about 20 to about 60 mg/mL). The of the described O-EDTA preparation would, in this example, constitute between about 1-10 mL (preferably about 2-3 mL) of the solution in a most particularly preferred embodiment of the flushing preparation.

Most preferably, the catheter will be flushed with a volume of about 3 mL of the described O-EDTA preparation containing about 30 mg/mL EDTA and about 3.5 millimolar n-octanoic acid. The catheter is to be flushed periodically at intervals of once a week, once every 4 days, once every 2 days, once a day, twice a day, every four hours, or as needed according to patient needs, with between about 2-3 mL of the O-EDTA preparation. The catheter flushing regimen may simply constitute once every time that the catheter is changed. In a preferred aspect of the method, the catheter is to be flushed at 4 hour intervals with the herein described preparations.

The compositions describe herein preferably remain therapeutically effective for use as a catheter-flushing agent after storage at a refrigerated temperature. However, the O-EDTA solution should be brought to room temperature before use on an animal or patient.

The present invention in still another aspect provides a kit. In one embodiment, the kit comprises a container, such as a syringe, holding a volume of one of the foregoing solutions containing a C₄-C₉ carboxylate antimicrobial agent and a chelating agent, anticoagulant or antithrombotic agent and an implantable catheter lumen to receive the solution. The kit may further comprise a package, such as a box, tray, tube, envelope, pouch, or the like, for holding the container. The volume of the solution in the container is typically in the range from 1 mL-20 mL, preferably from 2 mL-10 mL, usually being about 2 mL-4 mL. Optionally, the container will usually comprise a syringe, or device to permit direct introduction of the solution into the indwelling catheter.

In one embodiment, the kit comprises a container, such as a compartmentalized syringe, that comprises a plurality of compartments. For example, the container can have three compartments, where one compartment comprises a C₄-C₉ carboxylate antimicrobial agent, such as octanoic acid; the second compartment comprises a chelating agent, such as EDTA, an anticoagulant or an antithrombotic agent (e.g., heparin or hirudin); and the third compartment comprises a diluent, such as saline, Ringers solution, or water pH adjusted to 5.2 or less. Kits that include a carrier adapted to receive at least two compartments constitute still another embodiment of the kit. In these embodiments, the chelating agent, antithrombotic agent, or anticoagulant, would be included together with the C₄-C₉ carboxylate antimicrobial agent within a compartment of the container. The second compartment would comprise a diluent, such as the ones described above. In a preferred aspect, the chelating agent and antimicrobial agent are included together in a compartment of the device in dry powder form. The dry components would preferably be combined with the diluent of the second compartment to provide a solution suitable for use.

In these various embodiments, the kit preferably includes a chelating agent. In particular embodiments, the chelating agent is EDTA, and the C₄-C₉ carboxylate antimicrobial agent is preferably an antimicrobial. By way of example, such an antimicrobial is n-octanoic acid.

In yet another aspect of the present invention, a method for disinfecting an implanted catheter is provided that includes introducing a solution comprising a C₄-C₉ carboxylate antimicrobial agent and a chelating agent, anticoagulant or antithrombotic agent, in a pharmaceutically acceptable carrier solution into a lumen of a catheter where at least a portion of the catheter is sufficiently porous to permit diffusion of the solution outwardly from the lumen to the outer surface of the catheter and into the tissues or the bloodstream surrounding the catheter to inhibit infection. The implanted catheter may be a subcutaneous or transcutaneous indwelling catheter.

The ability to inhibit or prevent infection of the implanted catheter can be improved by utilizing catheters where at least a portion of the catheter body is sufficiently porous to allow the antimicrobial locking solution to permeate the catheter body and, preferably, pass outwardly (i.e., seep, ooze, leak, diffuse) into the tissue region surrounding the catheter. While the use of such porous or partially porous catheter bodies can be beneficial with many antimicrobial locking solutions, such as those taught in U.S. Pat. Nos. 4,186,745; 4,767,400; 4,968,306; 5,077,281; 5,913,856; 6,949,087; 7,004,923; and U.S. Patent Publication Nos. 2006/0074388 and 2006/0253 101, it is particularly useful with the acids of the present invention. It will be appreciated that C₄-C₉ carboxylate antimicrobial agents have molecular weights and other qualities which enable them to readily penetrate into and through many porous materials. Exemplary porous materials for construction of the catheter body include silicone rubber, expanded PTFE (e.g., GORE-TEX®, medical membranes), Teflon® films, natural, regenerated or semi-synthetic cellulosic materials such as cellulose acetate, cellulose diacetate, cuprophane, and the like. Such materials may be formed into the tubular catheter bodies or may be incorporated as separate component(s) into the catheter bodies.

The described compositions and preparations are expected to be effective in preventing the adherence and colonization of catheter surfaces by S. aureus, S. epidermidis, and fungi, as well as effective in both treating and eliminating already formed glycocalyx formations of these infectious organisms.

It is contemplated that whenever appropriate, any embodiment of the present invention can be combined with one or more other embodiments of the present invention, even though the embodiments are described under different aspects of the present invention.

### BRIEF DESCRIPTION OF THE FIGURES

FIGS. 1A and 1B illustrate methods according to the present invention for locking and disinfecting a transcutaneous catheter.

FIGS. 2A-2C illustrate methods according to the present invention for flushing, locking and disinfecting a subcutaneously implanted catheter.

FIGS. 3A-3C illustrate methods according to the present invention for flushing, locking and disinfecting a peritoneal dialysis catheter.

FIG. 4 illustrates an embodiment of the present invention where an antimicrobial locking solution permeates into an implanted catheter body and preferably into the tissue surrounding the catheter body.

FIG. 5 illustrates a kit constructed in accordance with the principles of the present invention.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

### DEFINITIONS

The terms below have the following meanings unless indicated otherwise.

The term “biofilm” as used herein refers to a polysaccharide-rich glycocalyx that typically accompanies microbial surface colonization.

As used herein, a “biofilm-resistant” device or surface is a surface or device that will prevent the adherence or growth of organisms that produce polysaccharide-rich glycocalyx material. Such organisms include, but are not limited to, the Staphylococcal aureus and epidermidis species.

The term “glycocalyx inhibiting concentration” as used herein refers to a concentration effective to degrade, dissolve, or otherwise inhibit a polysaccharide-rich glycocalyx. By way of example, such a polysaccharide-rich glycocalyx is characteristic of established staphylococcal infections of S. aureus and S. epidermidis.

By an “effective” amount or a “therapeutically effective amount” of a drug or pharmacologically active agent is meant a nontoxic but sufficient amount of the drug or agent to provide the desired effect, e.g., treatment of Staphylococcal and Candida infections. An appropriate “effective” amount in any individual case may be determined by one of ordinary skill in the art using routine experimentation.

As used herein, the terms “implanted”, “subdermal”, “subcutaneous” and “indwelling” are used synonymously to refer to medical device, e.g., catheter, placement. These implanted catheters typically will have a distal end which is at least partially open to a body lumen. Most commonly, the catheters will be intravascular catheters where the distal end is implanted in or attached to a blood vessel, usually a vein, but in some cases an artery. Exemplary intravascular catheters include hemodialysis and hemofiltration catheters, as well asintravenous catheters. Intravenous catheters can be used for a wide variety of purposes, including fluid infusion and drug delivery. Catheters attached other than to the vasculature include peritoneal dialysis catheters which are open to the peritoneal cavity and urinary catheters which open to the bladder.

The medical devices, such as catheters, which are described herein may be transcutaneously implanted or subcutaneously implanted. By “transcutaneously implanted,” it is meant that the distal end of the catheter is attached to or implanted within a target body lumen and a proximal end of the catheter is located externally to the patient. An intermediate portion of the catheter will thus pass through or penetrate the patient's skin, and the proximal end of the catheter will usually have a hub to permit selective attachment of infusion tubes, syringes, solution bags, and the like. Most commonly, the proximal attachment hub will have a luer fitting. By “subcutaneously implanted,” it is meant that the entire catheter is implanted beneath the skin and no portion of the catheter extends through the skin. Such subcutaneously implanted catheters are typically attached to a fully implanted hub at their proximal ends. The hub permits percutaneous access via a needle or other penetrating element.

Embodiments of the present invention provide pharmaceutically effective compositions of C₄-C₉ carboxylate antimicrobial agents in combination with chelating agents, anticoagulants or antithrombotic agents. These compositions are expected to be particularly useful in preventing the formation of the “biofilm” or polysaccharide-rich glycocalyx that typically accompanies microbial surface colonization. In particular, the compositions are expected to be most effective in breaking down staphylococcal glycocalyx and in inhibiting its formation. This feature renders the compositions of the present invention particularly useful in the treatment of staphylococcal infections where a polysaccharide-rich glycocalyx has formed or may potentially be formed, as well as in the prevention and treatment of Staphylococcal and Candida infection.

Embodiments of the present invention also provide treated or coated medical devices, such as catheters, that prevent staphylococcal or fungal colonization. The coating or film provided on these devices comprises a C₄-C₉ carboxylate antimicrobial agent, such as n-octanoic acid, and a chelating agent, antithrombotic agent or anticoagulant. A particular preferred combination of ingredients of the compositions includes n-octanoic acid and EDTA. Other preferred combinations comprise a glycocalyx inhibiting concentration or amount of a C₄-C₉ carboxylate antimicrobial agent and an anticoagulant or a chelating agent other than EDTA. Devices coated with these combinations of agents are also envisioned to be useful.

### C₄-C₉ Carboxylate Antimicrobial Agents

The C₄-C₉ carboxylate antimicrobial agents used in the compositions and methods described herein, include non-aromatic water-soluble C₄-C₉ alkyl, alkenyl or alkynyl organic acids, or mixtures thereof, or any of their water-soluble, pharmaceutically-acceptable salts. Such salts include, for example, sodium, potassium and ammonium salts. The sodium and potassium salts are preferred.

While the various carboxylate compounds exhibit different degrees of antimicrobial activity (per mole), the water-soluble n-alkane C₄, C₅, C₆, C₇, C₈ and C₉ carboxylates exhibit excellent antimicrobial activity. The n-hexanoic and n-octanoic acids and pharmaceutically-acceptable, water-soluble salts thereof are much preferred, with n-octanoic acid being more preferred. These materials in their free acid form rapidly kill essentially all important gram positive and gram negative pathogens, and Candida, at low solution concentrations in the acid pH range.

The microbiocidal activity of the C₄-C₉ carboxylate antimicrobials is directly related to the presence of their respective free acids in solution. The concentration of free carboxylic acid in solution, as opposed to carboxylate salt (anionic) form, is a function of the solution Carboxylic acid salts can be used, but only as long as the solution pH is such that a minimum lethal concentration (MLC) of free acid is present. Accordingly, the amount of acid or acid salt used will vary somewhat with the use pH. The amount of a given acid salt or acid that will provide the MLC at a given pH will depend on the pKₐ of the acid. Of course, knowing the pKₐ, the MLC of the particular acid and the use pH, the amount of any C₄-C₉ acid or acid salt to be used is easily calculated from the following formula

pKₐ=pH+log([HCₓ]/[C_{x-]),}

where [HCₓ] is the concentration of free acid of chain length x and [Cₓ₋] is the concentration of its anion.

Microbial testing with n-octanoic acid using the criteria described by Stone showed that a concentration of about 3 millimolar is the MLC for this acid. The MLC values described by Stone for n-butyric, n-pentanoic, n-hexanoic, n-heptanoic and n-nonanoic acids are as follows: C₄ (0.4 molar); C₅ (0.11M); C₆ (30 mM); C₇ (9 mM); C₉ (1 mM).

### The Second Agent

In addition to the C₄-C₉ carboxylate antimicrobial agents, the compositions and methods described herein also include a second agent selected from the group consisting of: (a) an anticoagulant, (b) an antithrombotic agent and (c) a chelating agent.

Examples of suitable chelating agents, anticoagulants, anti-thrombotic agents (including thrombolytic enzymes), as well as buffers that can be used in various embodiments of the present invention can be selected from Tables, 1, 2, 3 and 4, respectively.

TABLE 1
CHELATING AGENTS

Ascorbic acid
Deferoxamine
Dimercaprol
EDTA
Edetate Calcium Disodium
EGTA
DTPA
DMSA
Penicillamine
Succimer

TABLE 2
ANTICOAGULANTS

heparin
hirudin
acetylsalicylic acid
low molecular weight heparin
enoxaparin sodium
coumarin & indanedione derivative, anisindione
warfarin
protamine sulfate
streptokinase
urokinase
anti-thrombin III
atlephase recombinant, anistreplase
plasminogen activator

TABLE 3
ANTI-THROMBOTIC AGENTS

acetylsalicylic acid
dipyridamole
heparin
ibuprofen
indomethacin
prostaglandins
sulfinpyrazone
warfarin
Thrombolytic Enzymes
streptokinase
urokinase
plasminogen activator

TABLE 4
BUFFERING AGENTS

Ascorbate-Ascorbic acid
Acetate-Acetic acid
Citrate-Citric acid
Phosphate-Phosphoric acid
Tartrate-Tartaric acid
Malate-Malic acid
Fumarate-Fumaric acid
Malonate-Malonic acid
Barbiturate-barbituric acid

In certain preferred embodiments, the C₄-C₉ carboxylate antimicrobial agents are combined with EDTA. EDTA is available as calcium sodium EDTA and sodium EDTA formulations. The most preferred form employed by the present inventors is sodium EDTA. These formulations are provided at a concentration of 150 mg/mL.

In certain preferred embodiments, the C₄-C₉ carboxylate antimicrobial agents are combined with a chelating agent, such as ascorbic acid, that complexes calcium weakly, if at all. Where administration of too much locking solution or administration of the locking solution too quickly would produce calcium complexation leading to hypocalcemia potentially resulting in ventricular arrhythmias and sudden death, use of such a chelating agent can be desirable.

Table 5 provides a list of specific combinations of agents contemplated for use in the practice of embodiments of the present invention described herein.

TABLE 5
COMBINATIONS OF C₄-C₉ CARBOXYLATE ANTIMICROBIAL
AGENTS WITH CHELATING AGENTS, ANTICOAGULANTS
AND/OR ANTITHROMBOTIC AGENTS

EDTA + n-octanoic acid
EGTA + n-octanoic acid
Ascorbic Acid + n-octanoic acid
EDTA + C₄-C₉ carboxylate antimicrobial agents (e.g., n-octanoic,
n-butyric, n-pentanoic, n-hexanoic, n-heptanoic and n-nonanoic
acids)
EGTA + C₄-C₉ carboxylate antimicrobial agents (e.g., n-octanoic,
n-butyric, n-pentanoic, n-hexanoic, n-heptanoic and n-nonanoic
acids)
Ascorbic Acid + C₄-C₉ carboxylate antimicrobial agents (e.g.,
n-octanoic, n-butyric, n-pentanoic, n-hexanoic, n-heptanoic and
n-nonanoic acids)
Triethylene tetramine dihydrochloride (TTH) + C₄-C₉ antimicrobial
agents (e.g., n-octanoic, n-butyric, n-pentanoic, n-hexanoic,
n-heptanoic and n-nonanoic acids)
Hirudin + C₄-C₉ carboxylate antimicrobial agents (e.g.,
n-octanoic, n-butyric, n-pentanoic, n-hexanoic, n-heptanoic and
n-nonanoic acids)
Diethylene triamine pentaacetic acid (DTPA) + C₄-C₉ carboxylate
antimicrobial agents (e.g., n-octanoic, n-butyric, n-pentanoic,
n-hexanoic, n-heptanoic and n-nonanoic acids)
Diethylenetriamineacetic acid + C₄-C₉ carboxylate antimicrobial
agents (e.g., n-octanoic, n-butyric, n-pentanoic, n-hexanoic,
n-heptanoic and n-nonanoic acids)
Etidronate disodium (disodium salt of (1-hydroxyethylidene)
diphosphonic acid) + C₄-C₉ carboxylate antimicrobial agents (e.g.,
n-octanoic, n-butyric, n-pentanoic, n-hexanoic, n-heptanoic and
n-nonanoic acids)
Heparin + C₄-C₉ carboxylate antimicrobial agents (e.g.,
n-octanoic, n-butyric, n-pentanoic, n-hexanoic, n-heptanoic and
n-nonanoic acids)
Dimercaprol + C₄-C₉ carboxylate antimicrobial agents (e.g.,
n-octanoic, n-butyric, n-pentanoic, n-hexanoic, n-heptanoic and
n-nonanoic acids)
Citrate + C₄-C₉ carboxylate antimicrobial agents (e.g.,
n-octanoic, n-butyric, n-pentanoic, n-hexanoic, n-heptanoic and
n-nonanoic acids)
Methenamine + C₄-C₉ carboxylate antimicrobial agents (e.g.,
n-octanoic, n-butyric, n-pentanoic, n-hexanoic, n-heptanoic and
n-nonanoic acids)

As will be appreciated by those of skill in the art, the foregoing lists are only intended to be exemplary. Other chelating agents, anticoagulants and anti-thrombotic agents, as well as buffers, are also expected to be useful and effective in combination with a C₄-C₉ carboxylate antimicrobial agent. These combinations formulated as a coating will preferably further include a material, such as a cationic surfactant (e.g., tridodecylmethyl ammonium chloride or benzalkonium chloride), that will enhance adherence or film forming characteristics, of the preparation. As a solution for flushing or other medicinal use, the ingredients will be suspended in a carrier solution such as sterile saline, phosphate buffered saline, dextrose in water, Ringers solution, distilled water or any other physiologically acceptable solution pH adjusted to 5.2 or less.

### Methods of Flushing, Locking and Disinfecting a Catheter

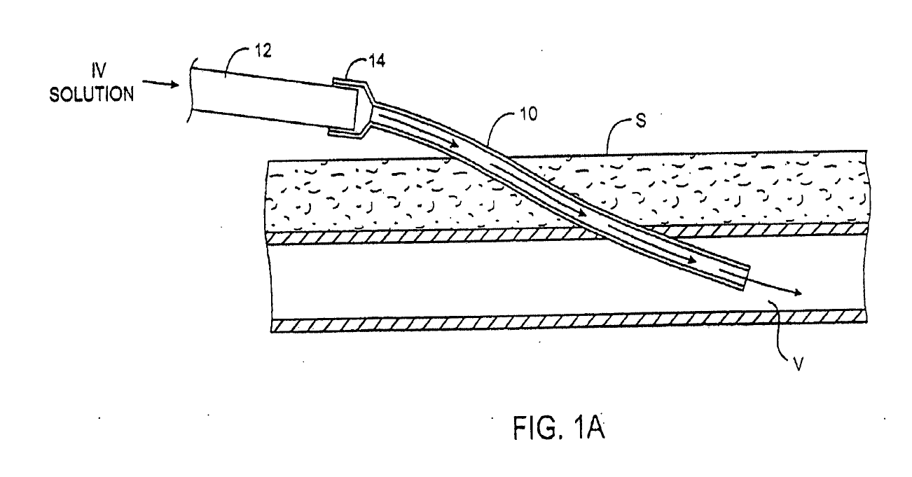

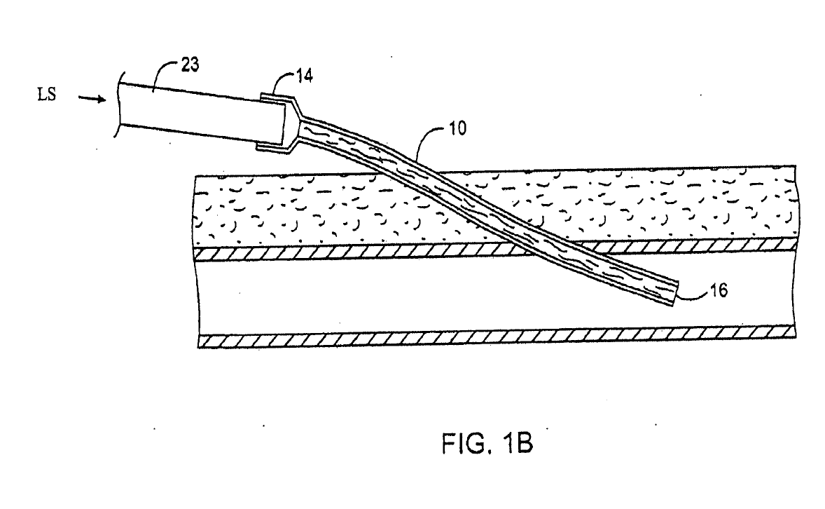

Referring now to FIGS. 1A and 1B, methods according to embodiments of the present invention for locking an implanted venous catheter 10 will be described. The venous catheter 10 will be implanted through a patient's skin S into a vein V for infusion of the patient. When it is desired to disconnect the patient from the source of infusion, it will be necessary to lock the catheter to inhibit plugging and fouling caused by coagulation, and preferably to further inhibit or eliminate the risk of infection. Shown in FIG. 1A, a tube 12 containing an IV solution will normally be connected to the proximal hub 14 of the catheter 10. The IV line 12 will be disconnected, and the catheter 10 rinsed with a flushing solution. After flushing is completed, a lock solution (LS) of a C₄-C₉ carboxylate antimicrobial agent and a second agent is introduced to fill the inner lumen of the catheter 10, as shown in FIG. 1B. Usually, a sufficient volume of the lock solution LS will be introduced to completely fill the lumen of the implanted catheter 10, with minimum excess passing from distal end 16 of the catheter. The loss of excess solution into a blood vessel or most other body lumens, however, will generally not be a problem. The “column” of the solution will then occupy the inner lumen, and the proximal hub will be sealed, helping retain the solution in place. The lock solution of a C₄-C₉ carboxylate antimicrobial agent and a second agent will effectively inhibit clotting and coagulation at the distal end 16 as well as inhibit or eliminate infection throughout the catheter. When it is desired to reattach the patient to the IV source, the solution will be removed and the catheter lumen flushed.

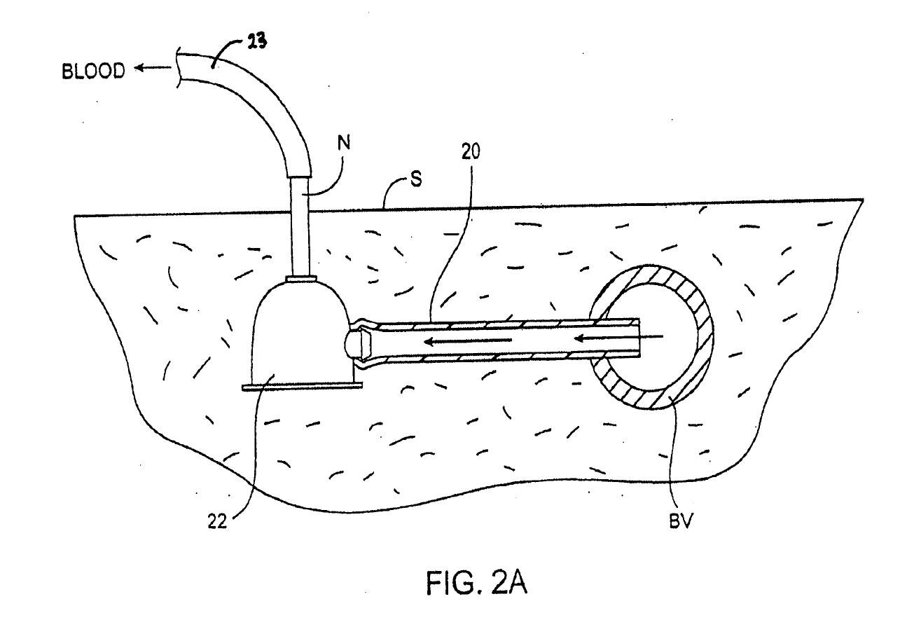

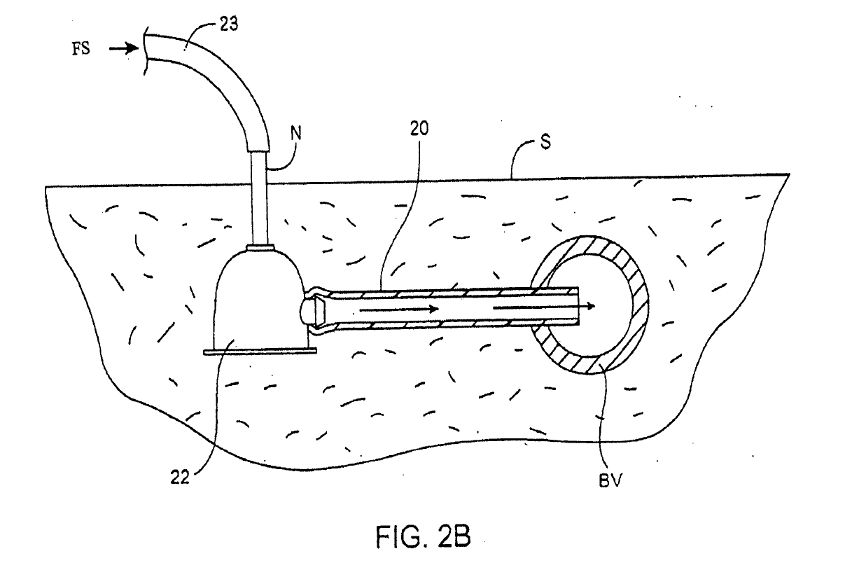

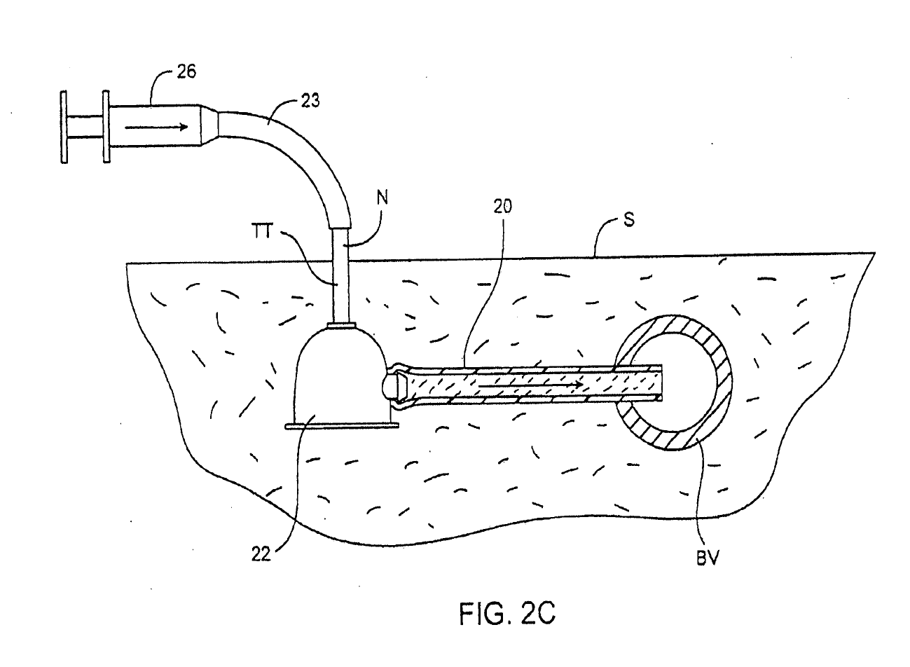

Referring now FIGS. 2A-2C, flushing and locking of a subcutaneously implanted catheter 20 used for hemodialysis access will be described. The catheter 20 is implanted between a target blood vessel BV, typically a vein, and an implanted port 22. During hemodialysis, blood is withdrawn through the catheter 20, through the port 22 and externally through a needle N and connecting line 23 used to percutaneously access the port 22 (FIG. 2A). Alternatively, the port and catheter can used to return treated blood to the patient.

When it is desired to end a hemodialysis (or hemofiltration) treatment, a flushing solution (FS) of a C₄-C₉ carboxylate antimicrobial agent and a second agent will be introduced through the needle N (typically from a syringe which is attached to the connecting line 23) to flush the lumen, as depicted in FIG. 2B. After the flush is complete, a container such as syringe 26 containing a lock solution is injected through the line 23/port 22 and into the lumen of catheter 20 to displace the flushing solution and lock the catheter (FIG. 2C). The lock solution will remain in place within the catheter 20. Alternatively or additionally, the lock solution can be a solution of a C₄-C₉ carboxylate antimicrobial agent and a second agent.

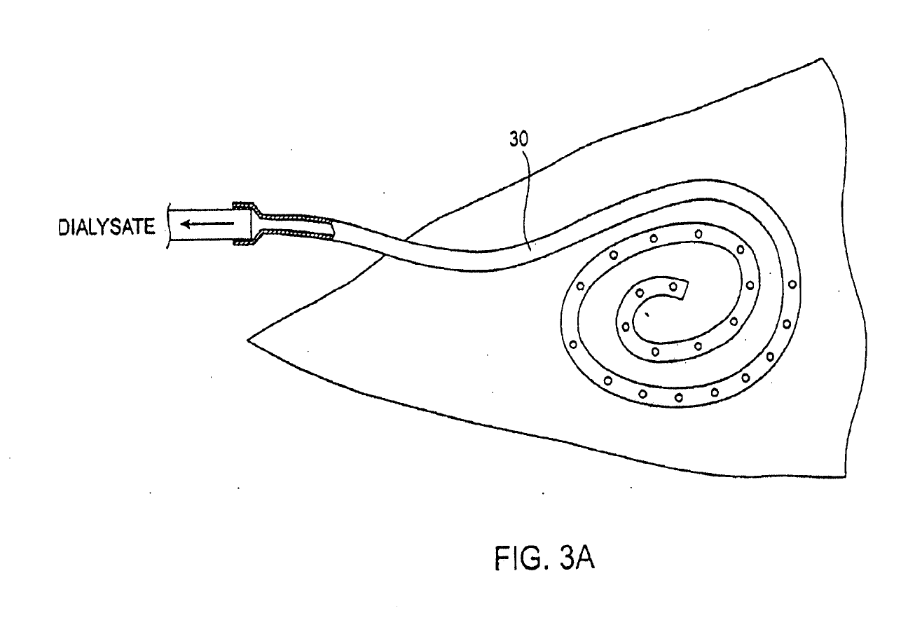

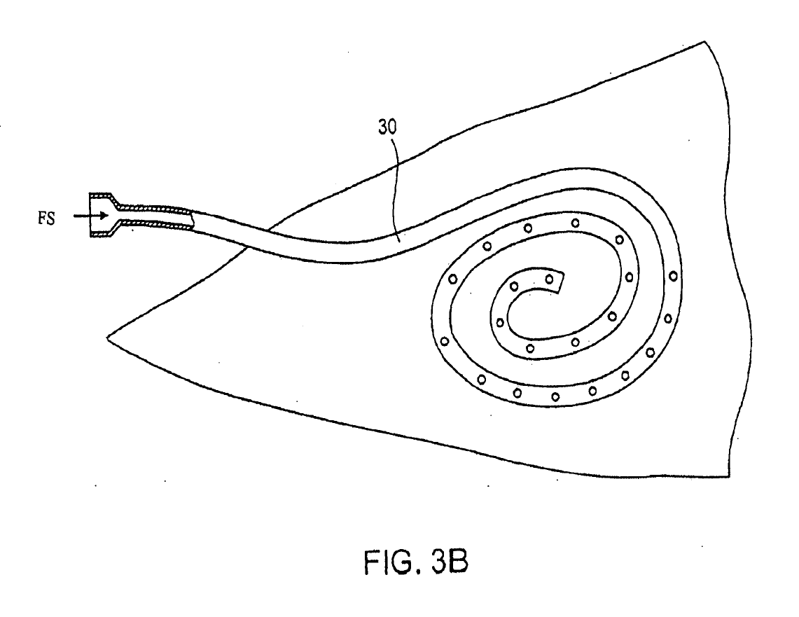

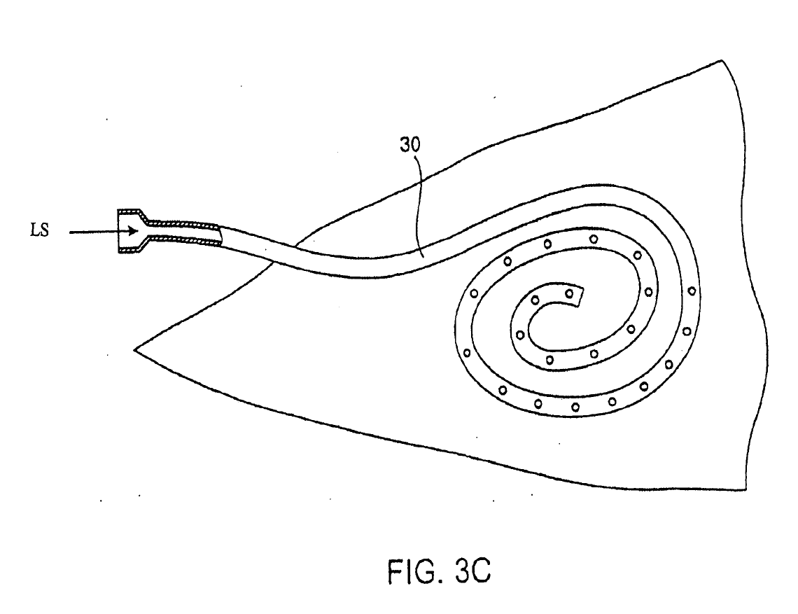

The methods of the present invention may also be used to flush and lock non-vascular catheters, such as peritoneal dialysis catheters 30, as shown in FIGS. 3A-3C. After a peritoneal dialysis treatment, the used dialysate is withdrawn from the catheter 30, as shown in FIG. 3A. After the dialysate has been sufficiently removed, the dialysis catheter 30 is flushed with a flushing solution FS of a C₄-C₉ carboxylate antimicrobial agent and a second agent, as shown in FIG. 3B. After flushing, the lock solution LS is introduced to the peritoneal dialysis catheter 30, as shown in FIG. 3C, so that it fills the lumen of the catheter, as described previously with the vascular catheters. Alternatively or additionally, the lock solution can be a solution of a C₄-C₉ carboxylate antimicrobial agent and a second agent.

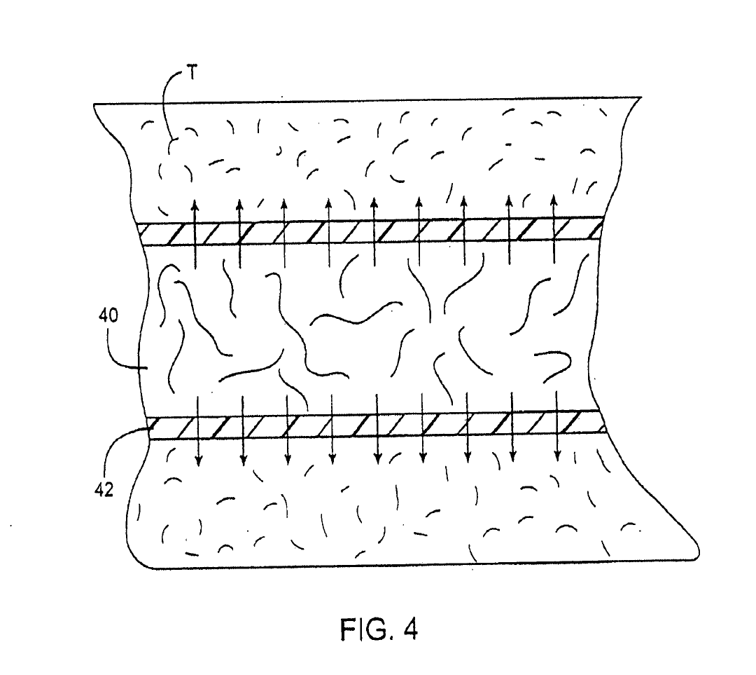

Referring now to FIG. 4, the use of a lock solution containing a C₄-C₉ carboxylate antimicrobial agent and a second agent can be enhanced by utilizing an implanted catheter which is formed at least partly from a porous material. When the lumen 40 of the porous catheter body 42 is filled with a containing a C₄-C₉ carboxylate antimicrobial agent and a second agent, the solution will be able to slowly penetrate (i.e., seep) into the catheter body and outwardly into the tissue T surrounding the catheter, as shown by the arrows in FIG. 4. Thus, the antimicrobial properties of the lock solution will not be entirely limited to the interior lumen of the catheter, but will also be effective on the surface of the catheter and in the tissue region immediately surrounding the catheter body. Particularly suitable materials and porosity properties for the catheter bodies have been set forth above.

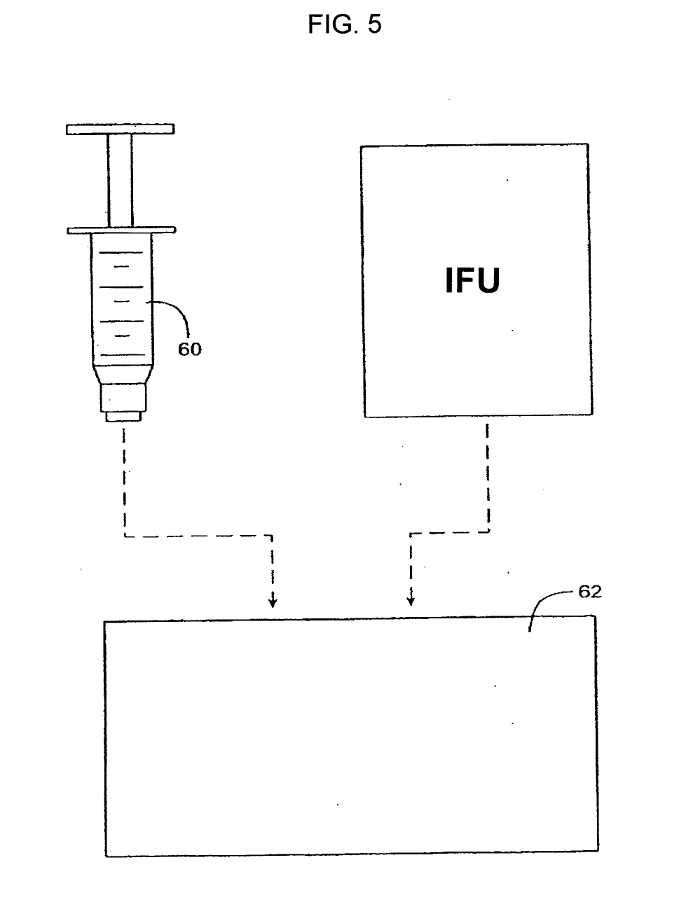

Referring now to FIG. 5, kits according to the present invention will comprise at least a container 60, such as a syringe, for holding a volume of a lock solution of a C₄-C_{y} carboxylate antimicrobial agent and a second agent and an implantable catheter lumen to receive the solution. The volume will typically be within the ranges set forth herein. The kits can further contain a package 62 to hold the container 60. The package can be any conventional medical device package, including boxes, tubes, envelopes, trays and pouches. In addition, the kit can contain instructions for use (IFU) setting forth a method for locking and/or disinfecting an implanted catheter by introducing the solution from the container into a lumen of the implantable catheter between successive uses of the catheter.

All references, patents, patent applications or other documents cited are hereby incorporated by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Preparation of an n-Octanoic acid-EDTA (O-EDTA) Pharmaceutical Composition

The present example describes the preparation of an O-EDTA pharmaceutical composition.

The n-Octanoic acid-EDTA (O-EDTA) solution was prepared to achieve final concentrations of about 3.5 millimolar octanoic acid and about 30 mg/mL EDTA in a sterile water solution. Separate solutions of EDTA (60 mg/mL) in sterile water and n-octanoic acid (7 millimolar) in buffer (pH-5) were prepared. The EDTA was reconstituted from EDTA powder (Sigma Chemical Co., St. Louis, Mo.). Octanoic acid was obtained from (Sigma Chemical Co., St. Louis, Mo.) and combined with a volume of buffer (pH-5) sufficient to constitute about 7.0 millimolar n-octanoic acid.

The 7.0 millimolar n-octanoic acid and 60 mg/mL EDTA solutions were mixed in equal volumes to constitute a 3.5 millimolar n-octanoic acid and 30 mg EDTA/mL buffered (pH-5) solution. The solution was stored in a sterile container.

Once formulated, the O-EDTA can be stored refrigerated at 4° C. until used. However, the composition should be brought to room temperature before administration to a patient or animal.

### Example 2

### Method for Maintaining Catheter Patency with an n-Octanoic Acid-EDTA Pharmaceutical Composition

The present example demonstrates one proposed embodiment of a method that may be used in maintaining the patency of an indwelling catheter in a patient. The regimen described herein is potentially applicable for use in both pediatric and adult patients. While the particular composition used was n-octanoic acid and EDTA the present example is applicable when using any of the combinations of n-octanoic, n-butyric, n-pentanoic, n-hexanoic, n-heptanoic and n-nonanoic acids antimicrobial agents and a chelating agent, anticoagulant or antithrombotic agent.

The particular dose of n-octanoic acid-EDTA in this regimen exposes patients only to relatively low, pharmaceutically acceptable levels of the EDTA and n-octanoic acid while providing effective infection control and catheter patency.

An indwelling catheter of a patient is flushed with a solution of n-octanoic acid/EDTA the “flushing” of the catheter constitutes filling the catheter with a volume of the n-octanoic acid-EDTA solution sufficient to provide a concentration of about 3 millimolar n-octanoic acid and about 90 mg of EDTA in the catheter. For a catheter volume of about 2-3 mL, the solution contains an EDTA concentration of between about 10 mg/mL-30 mg/mL. “Flushing” the catheter with about 3 mL of the O-EDTA solution thereby provides a dose of 0.5 mg n-octanoic acid and about 30-90 mg EDTA. The solution of n-octanoic acid-EDTA is prepared as described in Example 1.

“Flushing” of the catheter is achieved by adding between 2-3 mL of the n-octanoic acid-EDTA solution to the catheter. The solution is then allowed to diffuse throughout the catheter to the patient in which it is implanted. The concentration of the EDTA and n-octanoic acid in the solution is such that the patient will be exposed only to concentrations of the agents well below pharmacologically tolerable levels.

The flushing of the catheter is repeated at periodic intervals of once a week, once every 4 days, once every 2 days, once a day, twice a day, every four hours or as needed according to patient needs, to assure that infectious organisms are not allowed an opportunity to colonize the surface or initiate biofilm formation on the catheter surface.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. A composition comprising a C₄-C₉ carboxylate antimicrobial agent and a second agent selected from the group consisting of: (a) an anticoagulant, (b) an antithrombotic agent and (c) a chelating agent, wherein the agents are included in therapeutically effective amounts of between about 0.001 mg/mL and about 1000 mg/mL.

2. The composition of claim 1, wherein the second agent comprises a chelating agent.

3. The composition of claim 2, wherein the chelating agent is EDTA.

4. The composition of claim 2, wherein the chelating agent is selected from the group consisting of diethylenetriamine pentaacetic acid, Etidronate and EGTA.

5. The composition of claim 4, wherein the chelating agent is EGTA.

6. The composition of claim 2, further comprising an anticoagulant and/or an antithrombotic agent.

7. The composition of claim 1, wherein the second agent comprises an anticoagulant.

8. The composition of claim 7, wherein the anticoagulant is heparin or hirudin.

9. The composition of claim 7, wherein the anticoagulant is an anticoagulant other than heparin.

10. The composition of claim 1, wherein the C₄-C₉ carboxylate antimicrobial agent selected from the group consisting of n-alkyl C₄, C₅, C₆, C₇, C₈, and C₉ carboxylates.

11. The composition of claim 10, wherein the C₄-C₉ carboxylate antimicrobial agent is selected from the group consisting of n-octanoic, n-butyric, n-pentanoic, n-hexanoic, n-heptanoic, n-nonanoic acids and water-soluble, pharmaceutically-acceptable salts thereof.

12. The composition of claim 11, wherein the C₄-C₉ carboxylate antimicrobial agent is selected from the group consisting of n-octanoic, n-hexanoic, n-nonanoic and water-soluble, pharmaceutically-acceptable salts thereof.

13. The composition of claim 1, further comprising a pharmacologically acceptable carrier solution.

14. The composition of claim 13, wherein the pharmacologically acceptable carrier solution comprises saline, Ringers solution, or water pH adjusted to 5.2 or less.

15. The composition of claim 1, wherein the antimicrobial agent comprises a combination of a n-octanoic and n-heptanoic acids or water-soluble, pharmaceutically-acceptable salts thereof.

16. The composition of claim 1, wherein the antimicrobial agent is n-octanoic acid or a water-soluble, pharmaceutically-acceptable salt thereof.

17. The composition of claim 16, wherein the chelating agent is EDTA.

18. The composition of claim 1, wherein said chelating agent chelates Ca, Mg, Mn, Fe or Zn.

19. The composition of claim 1, wherein said chelating agent chelates calcium weakly, if at all.

20. The composition of claim 19, wherein the chelating agent is ascorbic acid.

21. A medical device coated with a composition comprising a C₄-C₉ carboxylate antimicrobial agent and a second agent selected from the group consisting of: (a) an anticoagulant, (b) an antithrombotic agent and (c) a chelating agent, wherein the agents are included in therapeutically effective amounts of between about 0.001 mg/mL and about 1000 mg/mL.

22. The medical device of claim 21, wherein the second agent comprises a chelating agent.

23. The medical device of claim 22, wherein the chelating agent is selected from the group consisting of EDTA, ascorbic acid, triethylene tetramine dihydrochloride, diethylenetriamine pentaacetic acid, Etidronate and EGTA.

24. The medical device of claim 23, wherein the chelating agent is EDTA.

25. The medical device of claim 23, wherein the chelating agent is ascorbic acid.

26. The medical device of claim 21, wherein the antimicrobial agent is n-octanoic acid or a water-soluble, pharmaceutically-acceptable salt thereof.

27. The medical device of claim 21, wherein the second agent comprises an anticoagulant.

28. The medical device of claim 27, wherein the anticoagulant is selected from the group consisting of acetylsalicylic acid, heparin and hirudin.

29. The medical device of claim 21, wherein the medical device is selected from the group of devices consisting of a central venous catheter, a peripheral intravenous catheter, an arterial catheter, a Swant-Ganz catheter, a hemodialysis catheter, an umbilical catheter, a percutaneous nontunneled silicone catheter, a cuffed tunneled central venous catheter and a subcutaneous central venous pert.

30. The medical device of claim 21, wherein the C₄-C₉ carboxylate antimicrobial agent is selected from the group consisting of n-butyric, n-pentanoic, n-hexanoic, n-heptanoic, n-nonanoic acids and water-soluble, pharmaceutically-acceptable salts thereof.

31. The medical device of claim 21, wherein the composition further comprises a pharmacologically acceptable carrier solution.

32. A process for coating a medical device comprising exposing the medical device to a composition for a sufficient amount of time to provide a coating on the exposed surface of the device, wherein the composition comprises a C₄-C₉ carboxylate antimicrobial agent and a second agent selected from the group consisting of a chelating agent, an anticoagulant, and an antithrombotic agent, wherein the agents are included in a therapeutically effective amount between about 0.001 mg/mL and about 1000 mg/mL.

33. The process of claim 32, further comprising treating the device with a surfactant before exposing the device to the composition.

34. The process of claim 33, wherein the surfactant is selected from the group of surfactant consisting of tridodecylmethyl ammonium chloride and benzalkonium chloride.

35. The process of claim 32, further comprising allowing the composition to dry on the surface of the device so as to form a film.

36. A method for locking and/or flushing an implanted catheter, said method comprising:
filling a lumen of an implanted catheter open to a body lumen with a locking and/or flushing solution comprising a C₄-C₉ carboxylate antimicrobial agent and a second agent selected from the group consisting of: (a) an anticoagulant, (b) an antithrombotic agent and (c) a chelating agent, wherein the agents are included in therapeutically effective amounts of between about 0.001 mg/mL and about 1000 mg/mL.

37. A method of disinfecting an implanted catheter; said method comprising:
introducing to a lumen of a catheter an antimicrobial solution comprising a C₄-C₉ carboxylate antimicrobial agent and a second agent selected from the group consisting of: (a) an anticoagulant, (b) an antithrombotic agent and (c) a chelating agent, wherein the agents are included in therapeutically effective amounts of between about 0.001 mg/mL and about 1000 mg/mL.

38. The method of claim 37, wherein at least a portion of the catheter is sufficiently porous to permit diffusion of the solution outwardly from the lumen to the outer surface of the catheter and into tissues or the bloodstream surrounding the catheter to inhibit infection

39. A kit for locking and/or flushing an implanted catheter, said kit comprising:
a container holding a volume of a solution comprising:
i) C₄-C₉ carboxylate antimicrobial agent; and
ii) a second agent selected from the group consisting of: (a) an anticoagulant, (b) an antithrombotic agent and (c) a chelating agent; and
an implantable catheter lumen which receives the solution.

40. The kit of claim 39, wherein said container comprises a syringe.

41. The kit of claim 39, wherein said container comprises a plurality of compartments.

42. The kit of claim 41, wherein one compartment comprises a pharmacologically acceptable carrier solution.
